(19) United States
(12) Patent Application Publication
(10) Pub. No.: US 2023/0026936 A1
(43) Pub. Date: Jan. 26, 2023

(54) Light Emission and Reflective Analyzing System

(71) Applicant: Matthew Kurlan, Encinitas, CA (US)

(72) Inventor: Matthew Kurlan, Encinitas, CA (US)

(21) Appl. No.: 17/870,669

(22) Filed: Jul. 21, 2022

Related U.S. Application Data
(60) Provisional application No. 63/224,251, filed on Jul. 21, 2021.

(51) Int. Cl. G01J 3/42

(52) U.S. Cl. CPC G01J 3/42; G01J 2003/423; A61B 5/0088

(57) ABSTRACT

A system employing projected light upon objects or surfaces to determine a safe light wave length range of light to be emitted for treatment of the object or surface is provided. Light emitters are employed for projecting light waves upon the surfaces along with light reception components which are employed to receive reflected light wave ranges from such emitted light. Thereafter, a safe light wave range can be employed to treat or affect the object or surface.

This application claims priority to U.S. Provisional patent application Ser. No. 63/224,251 filed on Jul. 21, 2021, which is incorporated herein in its entirety by this reference thereto.

### BACKGROUND OF THE INVENTION

### 1. Field of the Invention

The present invention relates generally to the employment of projected light to visualize and to interact with surfaces. More particularly, it relates to a device and method employing fiber optic or laser channelized frequency-specific light transmission and reflection reception therefrom, to both observe and affect a targeted surface or substance.

### 2. Prior Art

Materials exposed to light transmissions can either pass light through the material, when the material is substantially transparent, or reflect light from the material surface when the material is not transparent or they may absorb a portion of the spectrum and reflect a portion of the spectrum depending on the material. The reflection of transmitted light, contacting a material surface, occurs where the wavelengths of the light waves, striking a material surface, fail to match the vibration frequency of the molecules forming that object.

When natural or artificially generated light waves, in various light wavelengths strike an object, electrons in the atoms forming the object will vibrate. The electrons vibrate for brief periods of time with small amplitudes in their vibration whereupon the light energy is re-emitted from the material contacted, as a light wave. Where the material forming an object is transparent, these vibrations of the electrons forming it are communicated to other atoms forming the transparent material and then re-emitted on the side of the material opposite the side where the original light waves made contact.

Where the material forming an object is not transparent and is instead opaque, the vibrations of the electrons forming the material caused by the emitted light striking it, do not communicate through the atoms forming the material. In a material which is formed of molecules rendering it opaque, the electrons of the atoms struck by the emitted light on the material surface will vibrate for short periods of time, and then the energy in the light striking the material will form a reflected light wave.

This reflected light wave will be affected, as to color, by the vibration and the portions of the emitted light spectrum striking the object which are absorbed or otherwise affected by the vibration of the atoms forming it. Thus, the atoms of a material forming an object will vibrate when contacted with a light emission and will absorb portions or otherwise affect the color of the reflected light from the surface of that object. As such, the atoms and the material forming the object, can be determined by discerning the light frequencies or wavelengths reflected and those which have been absorbed or affected by the atoms of the material from which a reflected light is emitted.

With respect to the above, before explaining at least one preferred embodiment of the system herein, it is to be understood that the disclosed light emission and Analyzing device and system are not limited in application to the details of employment and to the arrangement of the components or the steps set forth in the following description or illustrated in the drawings. The light emission and reflection Analyzing system herein, and operations thereof disclosed, are capable of other embodiments, and of being practiced and carried out in various ways, all of which will be obvious to those skilled in the art once the information herein is reviewed.

Also, it is to be understood that the phraseology and terminology employed herein are for the purpose of description, and should not be regarded as limiting. As such, those skilled in the art will appreciate that the conception upon which this disclosure is based, may readily be utilized for other light generation and Analyzing systems. It is important, therefore, that the embodiments, objects and claims herein, be regarded as including such equivalent construction and methodology insofar as they do not depart from the spirit and scope of the present invention.

### SUMMARY OF THE INVENTION

In practical applications, the light generated and employed to effect photo-thermal reactions is conventionally laser or similarly generated. Such generated light may be at a wavelength which is arbitrary or which is calculated or convenient, depending upon the source element. However, emitted light of such a wavelength may fail to discriminate precisely between those substances which are intended to be affected by the light, so generated, and by those substances which are not.

The system and method herein disclosed and described takes advantage of the fact that specific differences in spectral absorption yielding reflected light may be invisible or hard to discern by the naked eye. However, such spectral differences between the broadcast light waves to an object and the absorbed and thereafter reflected light therefrom, may be readily discerned through the employment of a spectrophotometer.

By comparing the differences in light frequency or wavelength, between an originally emitted light communicated upon an object and the wavelength or frequency of reflected light therefrom, an absorbed wavelength can be determined. Thereafter by tuning of the wavelength of the light emissions from the source light accordingly, these light wavelength or frequency differences between the emitted light, absorbed light, and reflected light, may be utilized to more specifically target molecules which have absorbed light waves which were not reflected. Such a calculation can yield an identification of substances which are desired to be made to undergo a photo thermally induced reaction while leaving adjacent substances unaffected. Further, such a photo thermally induced reaction can be very specific as to a specific material to be affected, while leaving those adjacent substances, which are not intended to undergo such a reaction, substantially unchanged.

As such, by employing such a tuned light generation and light reflection system, to identify multiple adjacent substances on a surface, targeted substances can be identified and adjacent or background substances from those intended to be affected, can be identified to be preserved. Thereafter, once a calculation has been determined for light waves which will affect the targeted substances only, the targeted substances may undergo a photo-thermal reaction or vaporization at a precisely selected wavelength which has been determined by discerning the light waves reflected by the targeted substance from those which have been absorbed.

In medical applications of the system herein such a means, in real time, to determine specific light emissions which will affect a specific target material, while leaving adjacent material substantially unchanged, can significantly enhance the outcome of the medical procedure. Conventionally, such a spectral analysis has been limited to only identifying a material which has been extracted and then positioned physically into a remote spectral analyzing machine for analysis.

The system herein, through the employment of signal amplification technology of the device and system herein, can perform a comparable spectral analysis prior to and during a procedure. This is accomplished using light emissions which are transmitted a distance from a light emitting source, some of which are absorbed by the object and some of which are then reflected back from the object being analyzed, while the object is situated in an existing position or media.

Employing the disclosed device and system herein such spectral analysis, for one or a plurality of targeted materials, may be performed through the employment of fiberoptic light transmission and projection channels. Such will thus provide for real time material or tissue spectral analysis identification through the employment of such fiberoptic channels. This employment of fiberoptic channels for light transmission upon surfaces, which will both absorb and reflect the originally transmitted light waves and subsequent reception of reflected light waves not absorbed, will thus allow for analysis and treatment of living tissues, in real time, such as during surgery. By determining the frequency of light waves absorbed by a first material on the surface and the frequency of light waves which are reflected by an adjacent material on the surface, where the known spectrum of light waves have been communicated, a calculation can be made to determine an optimum treatment wavelength of light transmission to perform the intended task.

For example, this optimum treatment wavelength range of light transmission can be employed to destroy, remove, or alter a first tissue or material, while leaving the second, third, or other adjacent tissue or material substantially unaltered. In an example of use, which should be considered in no way limiting, direct light waves, at a known wavelength range, can be communicated, in an investigative emission, to the surfaces of teeth and adjacent gums having plaque and tartar at an intersection of the teeth and gums. The reflected light wave frequencies from each surface of a tooth, the adjacent gums, and the material forming tartar or plaque, from the initial investigative emission, will be received back by light sensors. A treatment or target light wave range can be determined from light wavelengths in the spectrum range broadcast in the investigative emission which have been absorbed by each tissue or material and those which have been reflected back to the light sensors. A determination of a target range of light waves reflected back from the target material of plaque and tartar can be determined by calculating the range of light waves from the emission striking it which are absorbed by the target plaque and tartar material.

While at this stage, the treatment device can be adjusted to emit only the determined target range of light waves to the teeth and gum surfaces, it is preferred that a secondary calculation be performed. This secondary calculation is performed to determine if any of the light waves in the determined target range of light waves overlaps with a light wave range which would be detrimental to the first surface of the gums and the second adjacent surface of the teeth.

Such a secondary calculation can be performed using a material database with stored safe light wave ranges which have been determined to be safe to gums and teeth, and damaging light wave ranges, which have been determined to potentially damage gums and teeth. In this secondary calculation, by comparing the determined target light wave range to the determined damaging light wave ranges in the database, any overlapping light waves can be ascertained where potential damage can be caused by the first determined target light wave range. These overlapping light wave ranges will then be removed from the first determined target light wave range to thereby determine a safe target light wave range for treatment. Thereafter, treatment can proceed using a projection of light from a laser or fiberoptic emitter or the like, which is projected onto the teeth and gum intersection area only in the determined safe target light wave range.

In this non limiting example, the system in real time would be effective to remove or destroy the plaque and tartar but have no or little effect on the adjacent gums and teeth. Such could be employed both for professional cleaning as well as with in-home cleaning devices akin to a toothbrush or water pick for dental hygiene.

Such a database for determined light waves, for example, can include determined inert light waves, such as a range in frequency in nanometers of light waves known to be substantially inert when striking a first surface, such as gums and a second surface such as teeth enamel. This database would also include a range in frequency in nanometers of light waves, which are absorbed or have a total internal reflection when contacting plaque or tartar and, as such, which will destroy it or render it to a material which will detach.

This example, as noted, should be in no way considered limiting. The same or substantially similar system can be employed, for example only, to determine a safe target light wave range to remove moles while leaving adjacent skin substantially unaffected or for surgery on tumors to remove or destroy a tumor while leaving adjacent healthy tissue substantially unaffected. Alternatively, in a non-tissue related mode of operation, the device and system herein can be employed for the cleaning of objects in a substantially similar mode of operation to determine the optimum safe light wave range for removing one substance while concurrently not affecting structures and material adjacent such. Such will also allow for the reduced invasiveness in many medical applications and in cleaning, and non medical application, it will speed up the process, for example, of cleaning and the like. Finally the device and system herein can save significant time through the provision of real time analysis of tissues, materials and substances to calculate a safe target light wave range of the spectrum, thereby increasing the effectiveness of medical procedures, as well as providing increased efficiency in industrial and processing of chemical substances.

With respect to the above description, before explaining at least one preferred embodiment of the herein disclosed light emission and analyzation system herein, it is to be understood that the invention is not limited in its application to the details of construction and to the arrangement of the components in the following description or illustrated in the drawings. The light emission and reflection Analyzing invention herein described, is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based, may readily be utilized as a basis for designing of other fiber optic channel light emission and Analyzing systems, and for carrying out the several purposes of the present disclosed container device. It is important, therefore, that the claims be regarded as including such equivalent construction and methodology insofar as they do not depart from the spirit and scope of the present invention.

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. Finally, the term “substantially” if not otherwise defined for size or dimension or positioning of a specific part or configuration, means plus or minus ten percent.

It is an object of this invention to employ real time light transmission and reflection analysis through fiber optic channels or similar light emission means to accelerate the analysis and safe treatment of living tissue during medical procedures.

It is a further object of this invention to employ such a system to reduce the invasiveness of medical procedures and to accelerate treatment of patients through substantially real time analysis of substantially safe light wave ranges for treatment.

Another object of this invention is the determination and employment of the appropriate safe light wavelength range for the cleaning or disinfecting of the substrate contacted thereby.

Other objects, features, and advantages of the present fiber optic channelized light transmission and analyzing system, as well as the advantages thereof over existing prior art, will become apparent from the description to follow and are accomplished by the improvements described in this specification and hereinafter described in the following detailed description which fully discloses the invention, but should not be considered as placing limitations thereon.

### BRIEF DESCRIPTION OF DRAWING FIGURES

The accompanying drawings, which are incorporated herein and form a part of the specification, illustrate some, but not the only or exclusive, examples of embodiments and/or features of the various modes of the fiber optic channelized light emission and Analyzing invention herein which as noted may be employed singularly or in combination. It is intended that the embodiments and figures disclosed herein are to be considered illustrative rather than limiting.

In the drawings:

FIG. 1 depicts a mode of the system herein employing a light emitter which is variable for frequency or wavelength ranges and depicts a computer interface for discerning the reflected light from the range projected from an object and the absorbed portion of the light.

FIG. 2 shows an example of software enabled analysis of a substance which may be affected through the discerning of the reflected light frequencies or wavelengths communicated through a fiber optic pathway to the spectroscopic analyzer.

FIG. 3 depicts an example of the determining of a light wave range transmission which may be generated by the light emitter which will target and affect a particular material or substance based on the calculation of FIG. 2.

FIG. 4 depicts an example of a treatment which may be provided by the system herein through the communication of light in safe light wavelength ranges calculated to only affect a targeted area or material upon which the wavelength or frequency-specific light is communicated, while concurrently leaving adjacent areas or tissues substantially unaffected.

### DETAILED DESCRIPTION OF THE PREFERRED EMBODIMENTS OF THE INVENTION

In this description, the directional prepositions of up, upwardly, down, downwardly, front, back, top, upper, bottom, lower, left, right, first, second, and other such terms refer to the device as it is oriented and appears in the drawings and are used for convenience only, and they are not intended to be limiting or to imply that the magnifying container device herein has to be used or positioned in any particular orientation.

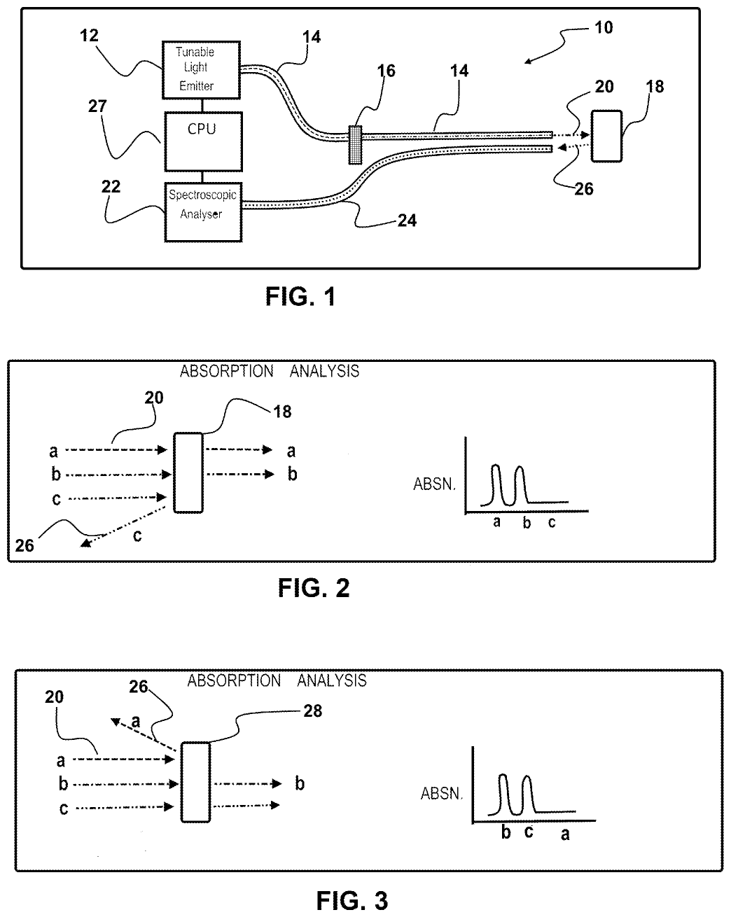

Now referring to drawings in FIGS. 1-4 wherein similar components are identified by like reference numerals, there is seen in FIG. 1 the system 10 herein in a graphic depiction of the components in general. As shown, a tunable light emitter 12 is operatively engaged with an output fiber optic conduit 14 which receives emitted light from the light emitter 12 and communicates such light in a wavelength range through the output fiber optic conduit 14. An optional filter 16 may be positioned along the output fiber optic conduit 14 between the light emitter 12 and the object 18 of interest. The filter 16 may be employed for filtering color or for polarization or for blocking a light wave range within that being emitted or for other purposes of the emitted light 20 from the light emitter 12.

Also shown in the system 10 in FIG. 1 is a reflected light sensor such as a spectroscopic analyzer 22 which is configured in operative engagement to a return fiber optic conduit 24. The return fiber optic conduit 24 communicates reflected light 26 from the object 18 when it is illuminated with a chosen frequency or wavelength of emitted light 20 from the light emitter 12. It should be noted, by the term “object” is meant one or each of a plurality of objects or surfaces which are adjacent to each other.

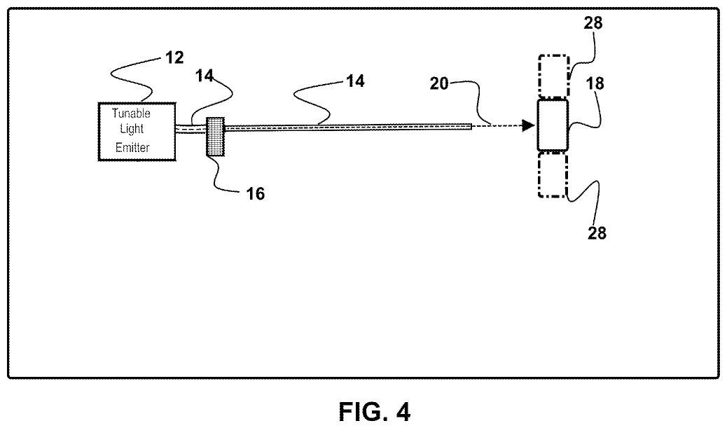

A CPU 27 is in operative electronic communication with both the spectroscopic analyzer 22 and the tuneable light emitter 12. The CPU 27 has a computer processor in operative connection to electronic memory in which analyzing software running to the task of receiving an electronic signal from the analyzer relating to reflected light 26 from the object to determine a light frequency or the wavelength range of absorbed light by the object, which has not been communicated to the spectroscopic analyzer 22 from the original range emitted and to the return fiber optic conduit 24. The determined target light wavelength range of the spectrum of the absorbed light, as noted herein, can be employed in a communication thereof to the object 18 to affect its structure while having substantially little or no effect on the surrounding area (FIG. 4). In a preferred secondary calculation, noted above, where adjacent tissue or structures are present with the targeted object, potentially damaging portions of the target light wavelength range can be determined which may affect the adjacent structure or structures. These damaging portions of the target light wavelength range can be removed and a safe target light wavelength range can be determined which will safely remove or otherwise affect the targeted object but concurrently, will cause substantially no damage or harm to any one or plurality of adjacent objects or surfaces.

For example and in no way limiting, the CPU and software running thereon to the task may determine a target light frequency or target light wavelength range from the light wavelength range which has not returned in a reflection from the target object 18 and into the return fiber optic conduit 24 and is therefor absorbed by the object 18.

In a subsequent step, such as in FIG. 4, the light emitter 12 will be tuned via an electronic signal by the CPU 27 to tune the emitted light 20 only to a spectrum, wavelength range, or light frequency range which was determined as absorbed. The emitted light 20, so tuned, would contact the object 20 and heat or otherwise affect its structure while, concurrently, having little or no affect on the surrounding area 28. Where the secondary calculation is performed, the emitted light 20, so tuned, will not project the emitted light 20 in any overlapping ranges between the targeted object 18 and any adjacent objects where it is known that such emitted light 20 in such overlapping ranges will cause damage or otherwise affect an adjacent object.

Also, while not depicted, should heat be generated by the system and process herein, it may require a conduit for cooling and/or removal of any vapors generated and emitted. Such a conduit would function best if positioned in proximity to where the light reacts with the targeted object 28 or substrate or substance. Thus, optionally, a means to remove excess heat and/or vapors, caused thereby, may be included. Such, for example, could be a conduit which would be adapted to provide a separate flushing and/or suction proximate to the position of reaction to the light transmitted for such.

Still further, optionally, a means to view the relative position of the distal end of the conduit communicating light for the operation herein may be desirable. For example only and in now way limiting, such may be provided by a metallic bead or some other structure viewable in an X-ray positioned at the light emitting tip, in order to allow for the use of fluoroscopy to determine a current position thereof during use.

Depicted in FIG. 2 is a graphic example of an absorption analysis to which the software running in electronic memory of the CPU 27 would be operating and configured to calculate. The CPU 27 will be in electronic communication with a database in electronic memory, which will enable the calculation. An electronic signal from the CPU 27 to the light emitter 12, in electronic communication with the CPU 27, will cause the light emitter 12 to adjust the spectrum or wavelength range of the emitted light 20 to the determined safe wavelength range to have the desired affect upon the object 18 when communicated through the output fiber optic conduit 14 to the surface of the object 18.

Additionally, a targeting camera (not shown but well known) can be operatively connected with the return fiber optic conduit 24 to provide a graphic depiction on a display screen of the object 18 in its remote position and for a subsequent targeting thereof.

FIG. 2 also shows an example of software enabled background material analysis by the software running in memory of the CPU 27 operating to such a task. As shown, any adjacent or surrounding material to the object 18, which may be affected by the emitted light 20, may be examined through the discerning of the reflected light 26 frequencies or wavelength range which is communicated through the return fiber optic pathway 24 to the spectroscopic analyzer 22 from the one or adjacent surfaces. As shown, emitted light 20 from the light emitter 12, in a first emission, strikes the background or surrounding areas 28 adjacent and around the object 18. The surrounding area 28 absorbs all the light in a wavelength range but for that designated as C. Since C is reflected by the surrounding area 28, a communication of emitted light 20 can be calculated in a safe wavelength range to the object 18 in the spectrum or wavelength range or frequency of C. Thus, the light emitted in a determined safe wavelength range may be employed to affect the object 18, but not the surrounding area 28.

In FIG. 3 is shown a graphic depiction of the software operating to the task of determining a light wavelength range or frequencies or spectrum which is reflected by the target or object 18 and what light wavelength range or frequencies or light spectrum has been reflected. The same can be determined for objects or surfaces or tissue or the like, adjacent the object 18.

As shown, emitted light 20 striking the target such as the object 18 and any adjacent surfaces, will reflect light in the spectrum, wavelength range, or frequency 26. A safe light wavelength range for communication to the target or object 18, thus, can be determined by the software operating to the task in electronic memory and on the CPU 27. Such, as noted, is accomplished by calculating the light wavelength range which has been both absorbed and reflected by each of any adjacent objects or surfaces, as well as the light wavelength range which has been absorbed by the object 18 from the light wavelength range or frequencies or spectrums of the first emitted light 20. A determined absorbed light wavelength range can be calculated as a range which will affect the target surface or object 18, and the light wavelength range of the returning reflected light 26 can be determined as not affecting the target or object and any adjacent surfaces.

Finally, depicted in FIG. 4 is an example of a treatment which may be provided by the system 10 herein, once the reflected light wavelength range and absorbed light wavelength range or spectrum has been determined for the target or object 18 using the system herein 10. As shown, the CPU 27 has communicated a signal to the light emitter 12, to only communicate emitted light 20 in a target light wavelength range of one or a plurality of light wavelengths in the range which has been calculated to be absorbed by the target or object 18, and reflected by the surrounding area 28. In this fashion, the target or object 18 can be affected by the emitted light 20 while protecting the surrounding area 28.

Additionally, in the secondary calculation, noted above, the absorbed light wavelength range, determined as having been absorbed by any adjacent object or surface, will be compared to the determined target light wave range. Should an overlap be determined in any of the absorbed light wave ranges and the target light wave range, the overlaps will be subtracted from the target light wave range for emission to a determined safe light wave range for communication to the target or object 18 which will have no affect on adjacent surfaces 28.

The description of the features of the light emission and reflective analyzing system invention herein does not limit the claims of this application, and other applications developed by those skilled in the art upon reviewing this application are considered to be included in this invention.

It is additionally noted and anticipated that although the depictions and disclosure herein is shown in its most simple form and operation, potential configurations, various components and aspects of the disclosed system may be differently arranged or slightly modified when forming the invention herein. As such, those skilled in the art will appreciate the descriptions and depictions set forth in this disclosure are merely meant to portray examples of preferred modes of the system herein within the overall scope and intent of the invention, and are not to be considered limiting in any manner.

Further, while all of the fundamental characteristics and features of the light emission system have been shown and described herein, with reference to particular embodiments thereof, a latitude of modification, various changes and substitutions are intended in the foregoing disclosure as well as the claims which follow, and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the invention as defined by the following claims.

What is claimed is:

1. An adjustable light emitting system for determining and generating a light wave range for communication to affect an object, as shown and described herein.
